(19) United States
(12) Patent Application Publication
(10) Pub. No.: US 2019/0232284 A1
(43) Pub. Date: Aug. 1, 2019

(54) POINT OF CARE ISOTHERMAL DIAGNOSTIC

(71) Applicants: Bruno L. Travi, Galveston, TX (US); Alejandro Castellanos, Galveston, TX (US); Scott T. Moen, Galveston, TX (US); Peter C. Melby, Galveston, TX (US); Omar A. Saldarriaga, Galveston, TX (US)

(72) Inventors: Bruno L. Travi, Galveston, TX (US); Alejandro Castellanos, Galveston, TX (US); Scott T. Moen, Galveston, TX (US); Peter C. Melby, Galveston, TX (US); Omar A. Saldarriaga, Galveston, TX (US)

(73) Assignee: The Board of Regents of the University of Texas System, Austin, TX (US)

(21) Appl. No.: 16/378,858

(22) Filed: Apr. 9, 2019

Related U.S. Application Data
(63) Continuation of application No. PCT/US2017/055513, filed on Oct. 6, 2017.
(60) Provisional application No. 62/406,242, filed on Oct. 10, 2016.

(51) Int. Cl. B01L 3/00; C12Q 1/686

(52) U.S. Cl. CPC B01L 3/502715; C12Q 1/686; B01L 2300/0861; B01L 2300/0816; B01L 2300/18; B01L 2300/047

(57) ABSTRACT

Certain embodiments are directed to a self-contained device and the use thereof for amplifying and identifying the presence of a nucleic acid with a specific sequence or sequences.

### PRIORITY INFORMATION

This Application claims priority to and is a continuation of PCT application PCT/US2017/055513 filed Oct. 6, 2017 and U.S. Provisional Application Ser. No. 62/406,242 filed Oct. 10, 2016, which are incorporated herein by reference.

### STATEMENT REGARDING FEDERALLY SPONSORED RESEARCH

This invention was made with government support under Grant No. UL1TR000071, awarded by the U.S. National Institute of Health under the Clinical and Translational Science Awards Program. The government has certain rights in the invention.

### DESCRIPTION

### I. Field of the Invention

The invention generally concerns a device and related methods for point-of-care-diagnostics. In particular the device and related methods are directed to a low-cost, field-applicable diagnostic device.

### II. Background

Infectious diseases have a massive impact on human health. Morbidity and mortality from infectious diseases particularly affect the world's poorest people (the so-called “bottom billion”). These diseases cause chronic disability with impaired development in children and reduced economic capacity in adults.

For example, leishmaniasis is a parasitic disease that may affect internal organs, such as the liver, bone marrow, and spleen, and may cause skin sores. The disease can be life threatening and may cause thrombocytopenia, anemia, and leukopenia. There are more than 1.2 million leishmaniasis cases per year (0.7 to 1.2 million cutaneous and 0.2 to 0.4 million visceral) and infections have been reported in more than 90 countries on five continents. 16% of the countries wherein leishmaniasis has been found are industrialized countries, 72% are developing countries, and 13% are among the least developed countries. 350 million people are at risk worldwide for leishmaniasis.

Malaria is another example, there are more than 190 million cases of malaria per year. In 2014 there were an estimated 214 million cases and 438,000 deaths from malaria. In 2015, 95 countries and territories had ongoing malaria transmissions. Approximately 3.2 billion people—almost half of the world's population—are living in areas at risk of malaria transmission. Sub-Saharan Africa (15 countries) continue to carry a high share of the global malaria burden, accounting for 88% of malaria cases.

Despite the huge burden of these diseases, diagnostic tests are not always available, particularly tests that are affordable and can be implemented in resource-poor regions of the world. Infectious agents are currently diagnosed using conventional tests developed for reference health centers or tertiary care facilities such as quantitative PCR, serology, and microscopy. These conventional tests require expensive equipment, trained personnel, and relatively complex laboratory facilities beyond the capability of health infrastructures of resource-limited endemic areas. For example, the diagnostic methods for malaria typically use rapid diagnostic tests (RDTs) that tend to have poor sensitivity when patients have low parasitemia. Diagnosis of leishmaniasis is based on PCR or antibody detection tests such as: rK30 and rK28 in ELISA or rapid immunochromatographic formats; Direct Agglutination Test (DAT); and immunoblotting. All of them carry significant disadvantages, such as the incapability to differentiate between clinically active and asymptomatic infections, and the inability to diagnose relapses as some of these tests remain positive for several months to years after cure. Molecular diagnostic tools like PCR and real-time PCR are sensitive and specific but are costly and require technological expertise. Detection of antibodies against the pathogens can be variable and persist after the pathogen is cleared.

There remains a need for additional point of care devices and methods for detecting pathogens or disease in resource challenged locations.

### SUMMARY

Described herein is a low-cost, field-applicable diagnostic device, in particular one that can be used at the point-of-care (POC) and is sensitive and specific. In certain aspects a device and methods described herein can be used to perform sensitive, field-applicable diagnostic tests that can be used at the POC. These sensitive diagnostic tests can lead to the reduction of disease burden by increasing the ability to diagnose carriers of the disease and direct treatment to more of those that need it and less to those that do not. Further, POC tests are considered to be indispensable tools for programs to eliminate specific diseases. The device and methods described herein can enable more accurate definition of the burden of disease in populations to directly inform those implementing disease intervention.

Embodiments described herein can enable the sensitive detection of specific or target nucleic acid (DNA or RNA), such as nucleic acid from a pathogen or a genetic disease marker. A sample can be processed using an amplification device comprising a sample inlet, an amplification reservoir, a reagent reservoir, a rehydration buffer reservoir, a product processing reservoir, a sample loading reservoir, and a detection region. In certain embodiments the detection region can be configured as a serpentine channel or be proceeded by a serpentine channel. In some embodiments, the device has the following advantages in addressing needs described above: (1) Nucleic acid amplification and detection can be performed in a self-contained closed system to minimize the risk of false positive tests that could result from contamination. (2) A device can be used without the need for sophisticated laboratory instruments or equipment and can be used in the field and/or at the point of care. (3) A device has the capability to use a number of different clinical specimens for the diagnostic test, including blood or any component of blood (liquid or dried), any body fluid or exudate, swabs of lesions or mucosal surfaces, tissue scrapings, tissue aspirates, tissue biopsies, urine, and feces. (4) A device can be a single-use disposable unit. (5) A device can be used by personnel with a minimal level of training. (6) The result of a test can be determined by visual inspection with the naked eye. (7) The technology can be more accurate than some detection techniques because it detects genomic material instead of an immune response to an antigen.

Certain aspects are directed to a self-contained device configured to amplify a target nucleic acid and indicate the presence or absence of the target nucleic acid in a sample, i.e., an amplification device. The device comprising a body that forms a plurality of reservoirs and fluid paths or channels connecting the reservoirs. The fluid paths or channels can be temporarily sealed or closed to separate one reservoir from another reservoir until the reservoir contents are needed in an adjacent or connected reservoir. The fluid path can be sealed by a frangible seal, a valve, or a constriction of the path.

In some instances, the device contains: an amplification reservoir, the amplification reservoir being configured to receive a sample through a sample inlet. The sample inlet can be sealed by a cap, a clip, a clamp, a plug, a one-way valve, or other mechanism. In certain aspects the sample is processed prior to introduction to the amplification device. Prior processing can include a number of methods including, but not limited to DNA extraction, sample purification, sample fractionation, and the like. The amplification reservoir can be configured as an amplification blister or chamber in that once it is desired to further process an amplification product the contents can be moved to other reservoirs downstream, by example applying pressure to the reservoir. In certain aspects pressure can be applied to the amplification reservoir, e.g., the amplification reservoir can be compressed, and the contents moved to another reservoir.

The amplification reservoir is fluidly connected with at least one reagent reservoir and at least one product processing reservoir. In certain aspects the reagent reservoir can contain one or more dry reagents. The reagent reservoir can be further connected to a rehydration buffer reservoir that contains a buffer or other solution for rehydrating the dried reagents in the reagent reservoir so that the reagent can be delivered to the amplification reservoir. The amplification reservoir is configured to receive a sample comprising a nucleic acid through a sample inlet to the amplification reservoir. Once the sample is in place the dry reagent can be rehydrated by transfer of the contents of the rehydration buffer reservoir, directly or indirectly (e.g., through a sample or other reservoir) to the reagent reservoir where the dried reagent is rehydrated. The reagent(s) can include, but are not limited to a nucleic acid polymerase enzyme, nucleotides, nucleic acid primers, and/or water. In some instances, the reagents are isothermal recombinase polymerase amplification reagents. The rehydrated reagent is then transferred to the amplification reservoir forming an amplification solution where target nucleic acids are amplified forming an amplification product. In certain aspects the amplification reservoir can also be a dilution reservoir. After an appropriate amount of time at an appropriate temperature the amplification product may or may not be transferred to a product processing reservoir that can in communication with a detection region/indicator strip. In an alternative embodiment the product can be transferred from the amplification reservoir to a loading reservoir where a selected volume of amplification product is collected and transferred to the product processing reservoir. This loading reservoir can be configured to control the amount of amplification product or sample volume introduced to the processing reservoir or the detection region. In another embodiment the loading reservoir is positioned between the product processing reservoir and a detection region so that only a selected volume of processed product is introduced to the detection region. In other embodiments a device can comprise two loading reservoirs, one prior to the product processing reservoir and a second loading reservoir after the product processing reservoir prior to the detection region.

The product processing reservoir is configured to receive the amplification product from either a loading reservoir or the amplification reservoir. The product processing reservoir can contain an indicator or probe capable of interacting with a target nucleic acid and identifying the presence of the target nucleic acid in the amplification product. The product processing reservoir can be interconnected with multiple loading reservoirs or amplification reservoirs.

Each of the reservoirs can be temporarily sealed by a cap, a frangible seal, a valve, or a constriction. In certain aspects, the frangible seal is capable of being broken by the application of pressure on the seal, e.g., the pressure can be a result of compressing a reservoir. In certain aspects the reservoir is configured as a blister. In other aspects the reservoirs can be temporarily sealed by valves, an external clamp, or a fold in the device. In some instances, the device is configured to be capable of providing a directional flow of a fluid within the device, permitting flow in one direction and/or preventing flow in another direction. In some instances, the fluid flow is restricted to one direction by selective manual pressure to the fluid, one-way valves, and/or blocking all but one pathway for a fluid from a particular reservoir. In some instances, a fluid path can be blocked by an external clip, clamp, or the folding the device to pinch and seal the fluid pathway. Each reservoir can be configured to hold a specific volume of fluid. Reservoirs of the invention are configured to have a volume ranging from 1, 5, 10, 20, 30, 40, 50, 60, 70, 80, 90, 100 μl or more.

In some aspects, the amplification reservoir is capable of receiving a sample containing a nucleic acid through a loading inlet. In some instances, the loading inlet comprises a luer-locked syringe adaptor, a screw-on cap, or a hinged snap cap. In some instances, the loading inlet is a rubber or polymer stopper or cap capable of being punctured by a syringe.

In some aspects, the indicator or probe is capable of indicating the presence of a target nucleic acid by visual detection. In some instances, a processed amplification product from the product processing reservoir or a loading reservoir can be transferred to or loaded on a lateral flow immunochromatographic or indicator strip. In certain aspects the amplification product is passed through a serpentine channel before, during, or after detection. In some instances, the indicator strip is housed in the device and is visible through a viewing window.

In certain embodiments the reservoirs can be aligned in a linear progression and connected by a fluid path. In certain aspects the body of the device is comprised at least partially of plastic. In certain aspects, the device can have a flex or bend portion or line at designated positions. In certain aspects the bend positions are configured so that when bent at a particular bend line the fluid path is constricted.

In other embodiments the device is a multilayer device comprising a support layer, a processing layer, and a top layer.

In certain aspects the device can further comprise or be configured to receive a sample from a sample processing device. In a further aspect the device can be configured to deliver to a processed sample to a detection device.

Methods of using the device are also disclosed herein. In some aspects, a method is disclosed for amplifying and identifying the presence of a target nucleic acid in a sample by: introducing a sample comprising a target nucleic acid into the amplification reservoir; combining the sample and a nucleic acid amplification reagent or nucleic acid amplification reagent mixture from a connected reagent reservoir forming an amplification solution. In some instances, amplification reagents, e.g., the nucleic acid polymerase, nucleic acid primer, nucleotides, and/or buffer, is in the reagent reservoir in a dried form prior to reconstitution with a rehydration solution. Prior to introduction into the amplification reservoir the amplification reagent(s) can be reconstituted or hydrated by application of a buffer or solution from a rehydration buffer reservoir connected to the reagent reservoir. A reconstituted nucleic acid amplification reagent or mixture can be introduced into the amplification reservoir to combine with the sample to form an amplification solution. In certain aspects the reagent delivered to the amplification reservoir can include, but is not limited to a nucleic acid amplification reagent or mixture. A nucleic acid amplification reagent or mixture can comprise a nucleic acid polymerase, a nucleic acid primer, nucleotides, a buffer, and water. The amplification solution can be incubated to allow amplification of target nucleic acids in the sample producing an amplification product.

Once the amplification solution has been properly incubated and an amplification product formed, the amplification product can be contacted with an indicator reagent. In certain aspects the amplification product is transferred to a product processing reservoir containing the indicator reagent. In certain aspects the amplification product can be transferred in part to a loading reservoir that is designed to control the amount of amplification product, volume of sample, or amount of amplification product and the volume being transferred to the product processing reservoir or the detection region. The indicator reagent can be comprised in a dilution buffer or other solution designed to facilitate detection of a target nucleic acid. In some instances, the method includes introducing the amplification product to the product processing reservoir where it is mixed with an indicator solution forming a detection solution. The detection solution is directly observed or can be introduced or loaded into or onto an indicator strip, where the presence of a target nucleic acid can be detected. In certain embodiments a selected volume of processed product is transferred to a loading reservoir downstream of the product processing reservoir and then introduced to the detection region.

Other embodiments of the invention are discussed throughout this application. Any embodiment discussed with respect to one embodiment or aspect of the invention applies to other embodiments or aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, devices, compositions, and kits of the invention can be used to achieve methods of the invention.

The use of the word “a” or “an” when used in conjunction with the term “comprising” in the claims and/or the specification may mean “one,” but it is also consistent with the meaning of ‘one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Reagent” or any variation of this term means a chemical substance or mixture for use in chemical analysis or chemical reactions. Non-limiting examples of reagents can include water, enzymes, buffers, nucleic acids, nucleotides, etc.

“Frangible” or any variation of this term means breakable through deformation. “Frangible seal” or any variation of this term means a seal that is breakable through deformation. Deformation can occur by any means known in the art. Non-limiting examples include applying an increase or decrease in pressure by bending, applying a fluid pressure, etc. For example, a frangible seal associated with a fluid path, reservoir, or blister can be broken by compressing a reservoir or blister and increasing the pressure on the seal.

The term “blister” refers to an enclosure formed by an outer covering that is raised at one or more faces or laterally extended forming a cavity or reservoir for housing a fluid. The blister is a reservoir used to retain a fluid or reagent until sufficient pressure is applied to the blister, forcing the contents to the next reservoir or compartment. The blisters can be made from a variety of materials, including without limitation mylar, polyvinyl chloride, thermoplastic materials, polyolefins, glycol-modified polyethylene terephthalate and combinations thereof.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 An example of one embodiment of DNA amplification card.

FIG. 2 An example of a method for amplifying DNA from a sample and testing for DNA identity using an embodiment of the DNA amplification card(s) in FIG. 1. Step 1. Open cap (101) and insert DNA sample into amplification blister (105). Step 2. Remove clamp (a) and push rehydration buffer in (103) to blister with dried reagents (104). Step 3. Remove clamp (b) and push mixture from blister with rehydrated reagents (104) to blister with sample (105). Step 4. Incubate mixture for a period of time for amplification. Step 5. Remove clamp (c) and push amplification blister (105) to product processing blister (106). Step 6. Push blister (106) to loading blister (108) and to indicator strip (109). Step 7. Detect bands in LF strip (109). The DNA-reagents mixture can be incubated at 40° C. for 30 minutes upon entering blister 105 where the sample has been introduced. In certain aspects blister 106 can be briefly massaged once it receives a small volume via loading reservoir 108 (2-6 μL) from blister 105. Then, approximately 100 μL of blister 106 can be moved into contact with the lateral flow strip. In an alternative embodiment a selected volume of processed product from 106 can be transferred to a downstream loading reservoir 108 and then contacted with the lateral flow strip. In one embodiment, the results can read in approximately 10 minutes.

FIGS. 3A-3B. Illustrates an alternative embodiment of a DNA amplification card. FIG. 3A is a line drawing of a top view of the DNA amplification card; FIG. 3B is a side perspective space filling view of the DNA amplification card.

FIG. 4. Is a line drawing illustrating a top view of the DNA amplification card with the clamps removed.

FIGS. 5A-5B. Illustrates a flexible sample apparatus of the DNA amplification card illustrated in FIGS. 3A-3B. FIG. 5A is a line drawing illustrating a top view; FIG. 5B is a space filling drawing illustrating a side perspective view.

FIG. 6 An example of one embodiment of a DNA extraction card.

FIG. 7 An example of a method for isolating DNA from a sample using a DNA extraction card. 1. Insert sample to sample blister containing lysis buffer. 2. close cap and incubate sample with lysis buffer. 3. Move the clamp/clip to right to open the channel from the blister containing lysis buffer (channels represented by (a)) and squeeze (push or fold or roll up) blister with sample to move the sample in lysis buffer to the DNA binding column. 4. Move the clamp/clip to right to open the channel (a) from the blister with washing buffer (b) and squeeze (push or fold or roll up) blister with washing buffer to wash the DNA binding column. 5. Move the clamp/clip to right to open channel (a) from the blister with elution buffer (c) and squeeze (push or fold or roll up) blister with elution buffer (c) to elute DNA from the DNA binding column. The eluted DNA can be collected and/or loaded onto any one of the nucleic acid amplification and indicating device disclosed herein.

FIG. 8 Illustrates another embodiment of the invention that incorporates a serpentine channel.

FIG. 9A-9B Illustrates another embodiment of the invention.

FIG. 10A-10B Analytical sensitivity of RPA-LF to detect Trypanosoma cruzi. Tenfold serial dilutions of parasite DNA were extracted with Qiagen DNeasy blood and tissue kit, and used to develop a standard curve upon amplification by qPCR (SYBRgreen; gold standard) (A). The same DNA dilutions were used to determine the limit of detection of RPA-LF (B). w, water (negative control). The control band in the LF strip is the upper band, while the test band is the lower band. qPCR, quantitative PCR; RPA-LF, recombinase polymerase amplification-lateral flow.

FIG. 11A-11B Specificity of RPA-LF and capacity to amplify all DTUs of T. cruzi. Species of Trypanosomatidae (Leishmania amazonensis, Leishmania mexicana, and Leishmania braziliensis), Plasmodiidae (Plasmodium vivax and Plasmodium falciparum), or human DNA were not amplified by the RPA-LF test (A). All DTUs (I-VI) produced strong bands in the LF strip using equivalent DNA input for RPA amplification in all the samples (B). DTUs, discrete typing units; NTC, no template control.

FIG. 12 Specificity of RPA-LF showing no cross-reactivity with Leishmania infantum (Leishmania chagasi). RPA-LF, recombinase polymerase amplification-lateral flow.

### DESCRIPTION

Certain embodiments are directed to a self-contained device and methods of using the device for amplifying and detecting a target nucleic acid. In certain aspects the target nucleic acid is a portion of a genomic nucleic acid. In a further aspect the genomic nucleic acid is DNA or RNA. In still a further aspect the genome is a bacterial, viral, parasite, vertebrate, mammalian, or human genome. In certain aspects the device can detect one or more infections (parasitic, bacterial, fungi, or viral), and ideally, can distinguish among taxonomically similar microbes. In a further aspect the device can be used to identify co-infection where disease prevalence overlaps. Certain embodiments are directed to devices for identifying the presence of an organism or disease by analysis of a biological sample including, but not limited to Leishmania, Plasmodium (malaria), T. cruzi (Chagas), Giardia, Cryptosporidium, Entamoeba, Fasciola, Strongyloides, Zika, Dengue, and/or Chikungunya to name a few. These organisms can be detected in blood, cutaneous tissue, stool, or fractions thereof.

### I. AMPLIFICATION DEVICE

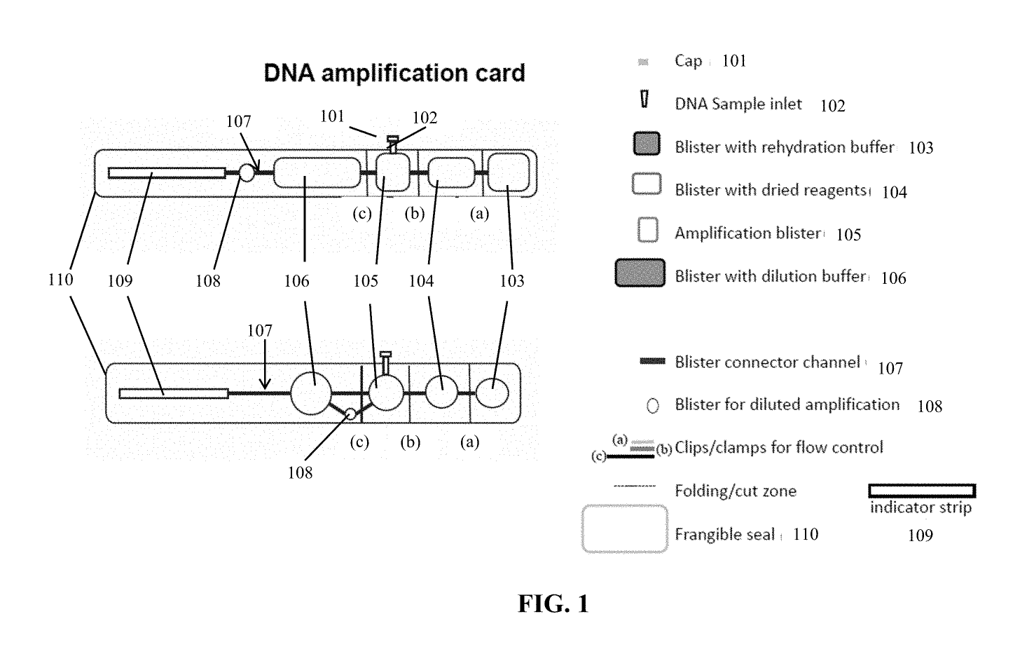

Embodiments of an amplification device are illustrated in FIG. 1 to FIG. 5. Referring to FIG. 1, the device can comprise a body that forms a plurality of reservoirs and fluid paths or channels 107 connecting the reservoirs. The fluid paths or channels can be temporarily sealed or closed to separate one reservoir from another reservoir until the reservoir contents are needed in an adjacent or connected reservoir. The fluid path can be sealed by frangible seal or a constriction of the path indicated by item a, b, and c of FIG. 1. The device can include sample inlet (102), amplification reservoir (105), reagent reservoir (104), rehydration buffer reservoir (103), product processing reservoir (106), an optional loading blister(s) (108), and detection region (109). Cap 101 can be seal or cover a DNA sample inlet (102) that is fluidly coupled to amplification blister (105). The rehydration buffer reservoir (103) can be fluidly coupled to reagent reservoir (104). In certain aspects reagent reservoir (104) can contain dried reagents. Reagent reservoir 104 is also fluidly coupled to amplification reservoir (105). Amplification reservoir (105) can be coupled to product processing reservoir (106), which is optionally coupled to an upstream and/or downstream loading blister (108). Loading blister 108 can couple amplification reservoir 105 to product processing reservoir 106 or product processing reservoir 106 to indicator strip (109). Loading blister 108 can be configured to modulate the amount of product or volume of processed product being loaded on the detection region 109.

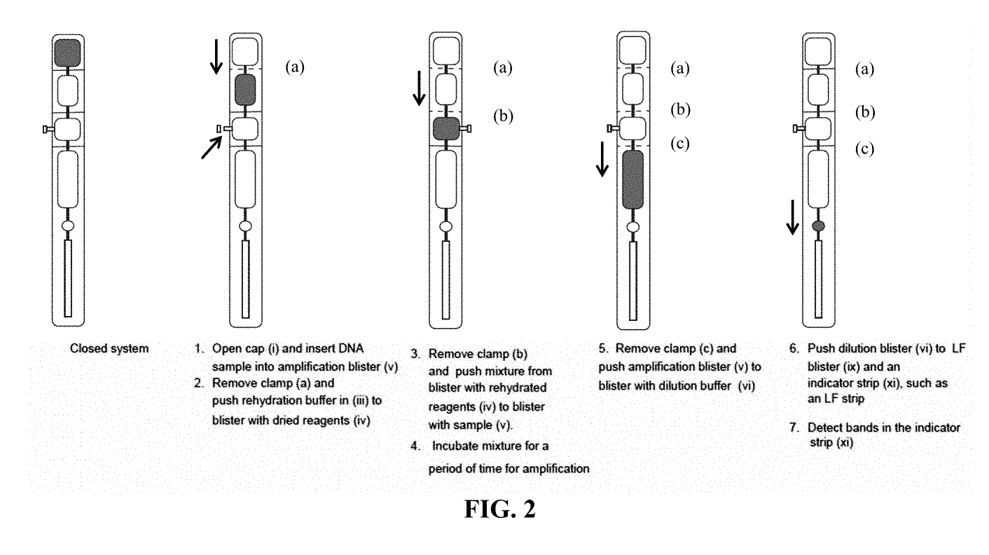

An example of how an amplification device described herein can be used is illustrated in FIG. 2. Referring to FIG. 1, the process can be initiated by opening cap (101) and inserting DNA sample into amplification blister (105). Removing clamp (a) and pushing rehydration buffer in (103) to reagent blister (104). Removing clamp (b) and pushing mixture from reagent blister (104) to amplification blister (105). Incubating the mixture for a period of time for amplification. Removing clamp (c) and pushing amplification blister (105) to product processing blister (106) or loading blister (108) and then to product processing blister (106), which can contain a diluting buffer. The diluted amplification product can be transferred from product processing blister (106) to loading blister (108) or to reagent detection region (109) by compressing product processing blister (106) or loading blister (108). Contents of loading blister (108) or product processing blister (106) can then be pushed onto and indicator strip (109). Amplification products can then be detected on indicator strip (109). In certain aspects an amplification reaction can be performed at a temperature of at least 30° C. to less than 100° C. for 10, 20, 30, 40, 60, 120, 240 minutes or longer. In certain aspects the reaction is performed at 60° C.

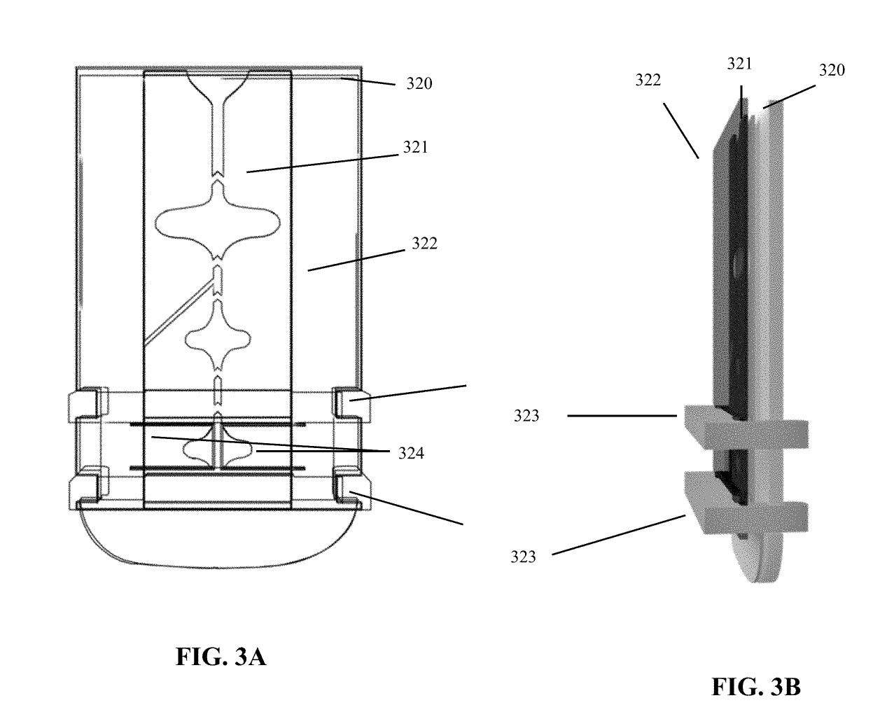

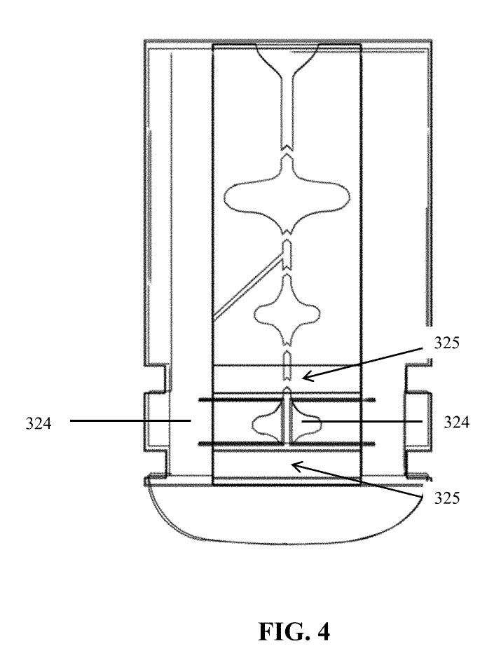

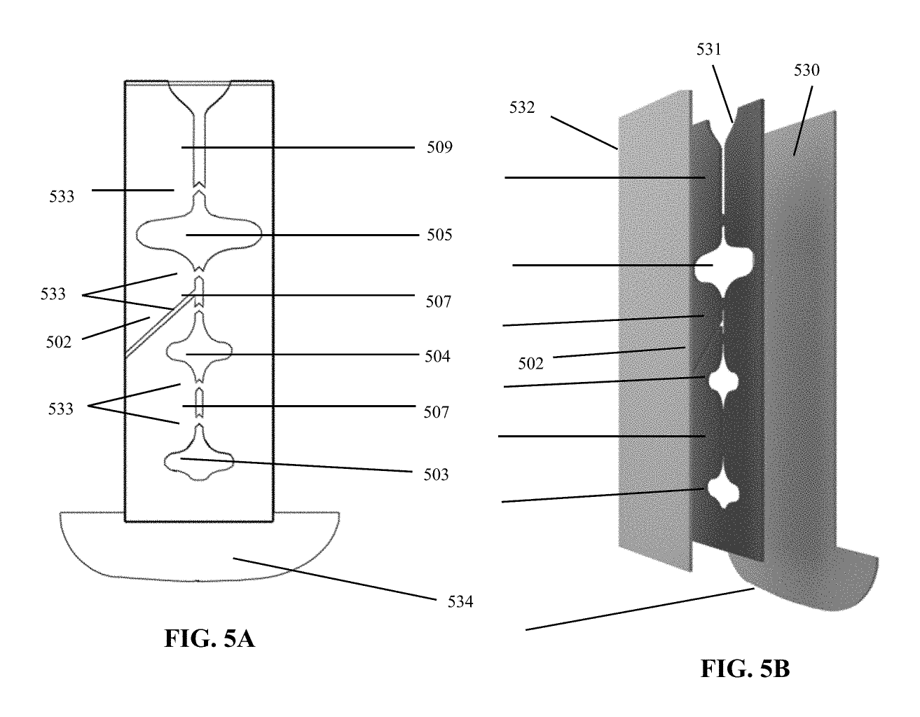

Another embodiment of a DNA amplification device described herein is illustrated in FIG. 3 to FIG. 5. This embodiment is a thin, inexpensive, chemistry mixing apparatus consisting of 3 layers—support layer 320, processing layer 321, and apparatus cover layer 322. The apparatus cover layer 322 can be made of a material that can confer mechanical forces to the solutions in the reservoirs or blisters of processing layer 321 without deformation. The support layer can be rigid and supports processing layer 321, which is further described below and in FIG. 5 in more detail. Processing layer 321 can include a pull-tab located on one end to assist pulling processing layer 321 when the apparatus in use. In certain aspects processing layer 321 can be pulled through mechanical resistance provided by hardware associated with the apparatus. In certain aspects the mechanical resistance can be provided by clip(s) 323 configured to contact and apply pressure to apparatus cover layer 322 at defined locations. In certain aspects the apparatus cover layer 322 can be configured to form an open notch that allows a protrusion of clip 323 to contact processing layer 321 and exert a mechanical force on processing layer 321. Apparatus cover layer 322 can also be configured to have tabs or mixing paddles 324 that can provide for increased flexing of the apparatus cover layer at desired location(s) to allow manual manipulation and mixing of the reservoir or blister contents positioned under mixing paddles 324. Clips 323 are configured to be removable and can be removed and replaced during use of the apparatus. FIG. 4 illustrates an apparatus with clips 323 remove exposing notches 325, also shown are mixing paddles 324. Processing layer 321, which contains the DNA amplification processing layer, i.e., contains the reagents, reservoirs, and fluid paths, without being bound by theory, can be made of double-sided pressure sensitive adhesive (PSA), spin-coated adhesive chemistry, or heat-welding of the first and second layer of processing layer 321. A unique characteristic of the reservoirs of this embodiment is that volume can be achieved primarily through lateral displacement in the X and Y axes (width and length). This design minimizes deformation in the Z axis (height) which can allow for the use of stiffer materials, transfer solutions when mechanical pressure is applied, and a greater surface area for mechanical mixing, e.g., finger perturbation.

FIG. 5 illustrates one embodiment of processing layer 321. The processing layer can include a first layer 530, reservoir layer 531, and a second layer 532. In certain aspects first layer 530 can be configured to have pull-tab 534 at one end of processing layer 321. First layer 530 and second layer 532 can be made of Mylar, or materials with similar characteristics, and can be coated an antifouling agent like polyethylene glycol (PEG) to prevent sample and reagent absorption to the internal walls of processing layer 321. In this embodiment processing layer 321 comprises a series of three reservoirs or blisters (503, 504, 505) connected by fluid channels (507) to the lateral flow device output 509 that can be either attached to, or on the device itself. The fluid paths or channels 507 can be temporarily sealed or closed to separate one reservoir from another reservoir until the reservoir contents are needed in an adjacent or connected reservoir. To separate reservoirs or blisters while the device is not in use, capillary valves 533 are placed or formed in the fluid channels 507 connecting reservoirs or blisters. These valves will be created, without being bound by theory, by thin chevrons of PSA or heat welding. Upon a predetermined threshold pressure exerted on the fluids in the apparatus, the valve will burst allowing the fluids to proceed to the subsequent reservoir or blister. With reference to FIG. 5, processing layer 321 can include sample inlet 502, amplification reservoir 505, reagent reservoir 504, rehydration buffer reservoir 503, an optional product processing reservoir, an optional loading reservoir(s) or blister(s), and detection region 509. A cap, plug, or constriction can seal or cover DNA sample inlet 502 that is fluidly coupled to amplification reservoir 505. The rehydration buffer reservoir 503 can be fluidly coupled to reagent reservoir 504. In certain aspects reagent reservoir 504 can contain dried reagents. Reagent reservoir 504 is also fluidly coupled to amplification reservoir 505. Amplification reservoir 505 can be coupled to product processing reservoir 506, which is optionally coupled to an upstream and/or downstream loading reservoir or blister. An optional loading reservoir or blister can couple amplification reservoir 505 to detection region 509. A loading reservoir or blister can be configured to modulate the amount of product or volume of processed product being loaded on the detection region 509.

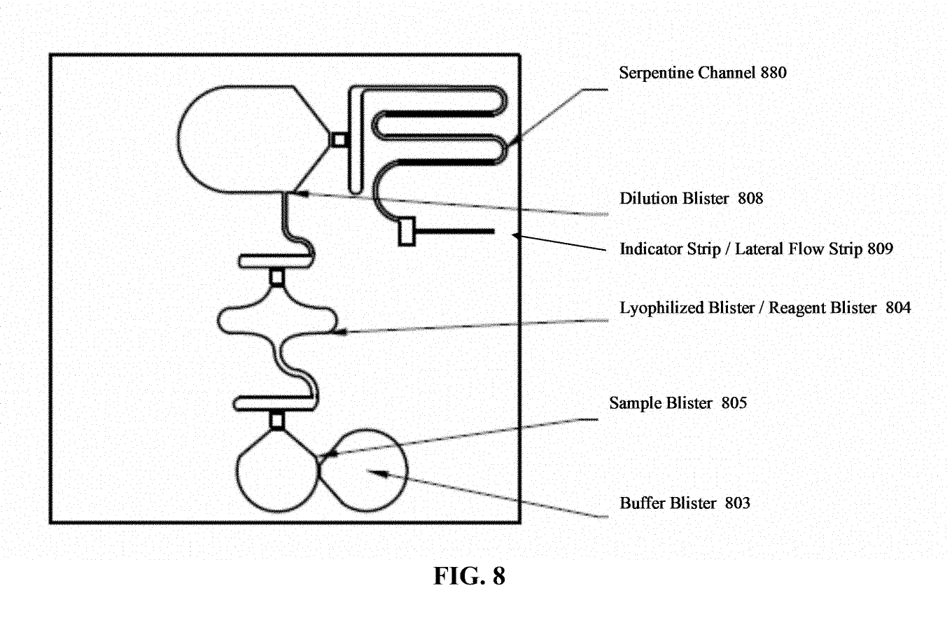

Referring to FIG. 8, the device can comprise a body that forms a plurality of reservoirs and fluid paths or channels connecting the reservoirs (e.g., blisters). The fluid paths or channels can be temporarily sealed or closed to separate one reservoir from another reservoir until the reservoir contents are needed in an adjacent or connected reservoir. The fluid path can be sealed by frangible seal or a constriction of the path. The device can include sample inlet that provide access to sample reservoir (805), amplification/dilution reservoir (808), reagent reservoir (804), rehydration buffer reservoir (803), an optional product processing reservoir, an optional loading blister(s), and detection region that can include a serpentine channel (880). Serpetine channel (880) can be coupled to an indicator strip (809). The rehydration buffer reservoir (803) can be fluidly coupled to sample reservoir (805) or alternatively to reagent reservoir (804). In certain aspects reagent reservoir (804) can contain dried reagents. Reagent reservoir 804 is also fluidly coupled to amplification/dilution reservoir (808). Amplification/dilution reservoir (808) can be coupled to product processing reservoir containing a serpentine channel (809), which can be optionally coupled to an upstream and/or downstream loading blister. A loading blister can couple amplification/dilution reservoir 808 to product processing reservoir or product processing reservoir to an indicator strip. A loading blister can be configured to modulate the amount of product or volume of processed product being loaded on the detection region.

In certain aspects the device is a handheld device having first dimension between 2 to 20 cm in length, a second dimension between 2 to 20 cm in length, and a height of 0.5 to 2 cm. In certain aspects the device is in the shape of a rectangular prism. In a further aspect the device is an elongated rectangular prism shape. The device can comprise a plurality of reservoirs interconnected directly or indirectly by one or more fluid paths or channels. The reservoirs can have a volume of 5, 10, 20, 30, 40, 50, 60, 70, 80, 90, or 100 μl or more, including all values and ranges there between. In certain aspects the reservoirs can be in the form of a blister. A blister is cavity or pocket formed by a flexible material that can be compressed or depressed to expel the contents of the cavity or the pocket.

In certain aspects the device can be made of one or more plastics or films. The body of the device can be rigid with flexible or bendable lines formed therein. The device can be a composite of two or more materials, such as a base made of a material suitable to forming reservoirs and a flexible film that is capable of keeping the fluid in a reservoir. The flexible film can form a blister compartment that is seal by a frangible seal, which can be broken by applying a force and allowing the contents of the blister to flow into an adjacent reservoir or channel. The flexible film can be capable of being manipulated so that the pressure in the reservoir is increased without bursting the flexible film. In certain aspects sufficient pressure breaks a frangible seal of the reservoir.

The device can be a self-contained device. The device can contain amplification reagents, detection reagents, and the like compartmentalized as needed. The self-contained device can have a sample inlet capable of receiving an outside sample containing nucleic acids. In certain aspects the sample inlet provides access to the amplification reservoir. The device can be capable of receiving an outside sample that is a liquid sample, a solid sample, or a dry sample. The sample inlet can be, but is not limited to, a luer-locked syringe adaptor, a manually removable screw-on cap, a hinged snap cap, a rubber or polymer stopper or cap, and/or a rubber or polymer stopper or cap capable of being punctured by a syringe.

The device includes multiple reservoirs. In some instances, the device body includes 2, 3, 4, 5, 6, 7, 8, 9, 10, 11, or more reservoirs. The reservoirs can be interconnected with other reservoirs in the device. Each reservoir can be interconnected to form a direct path where adjacent reservoirs are connected to an indirect path where there is an intervening reservoir. The reservoirs can be interconnected in series and/or parallel. The device can have some reservoirs that are interconnected in series and some that are interconnected in parallel.

The reservoirs can be interconnected through channels, tubes, ports, or directly to another reservoir. Movement of a fluid from one reservoir can be facilitated by applying pressure to the reservoir and/or using gravity. Pressure can be applied by, but not limited to, deforming the reservoir or inserting a plunger.

The flow of a fluid between reservoirs can be arrested or impeded by a seal; a kink in or compression of a fluid path; a valve; or the like. In certain aspect a seal can include, but is not limited to, a frangible seal. The flow of a fluid between reservoirs can be arrested by the blocking or compressing the path between reservoirs including, but not limited to, bending or clamping the path. The arrest or block of the fluid flow path can be reversible or permanent by application of pressure to a seal, physical movement of a seal, breaking a seal, dissolving a seal, clamping the path, or bending the path. A seal can be, but is not limited to a frangible seal, a removable plug, or a seal or blockage that can be removed or snapped off.

The flow of a fluid from a reservoir can be restricted to one direction or can flow in more than one direction. The flow can be restricted in one direction by the application of pressure in the same direction. One-way valves can be used to restrict flow in one direction. Flow can be restricted in one direction by the blocking of all other flow paths from or into a reservoir.

The reservoirs can be designed to hold a specific volume of a fluid or can be expandable. The reservoirs can be flexible or non-flexible. The reservoirs can contain a liquid or solution when the device leaves manufacturing. Some of the reservoirs can contain dry reagent material. Some of the reservoirs can be empty. The device can contain any combination of reservoirs containing a liquid or a dry reagent, or empty reservoirs prior to use.

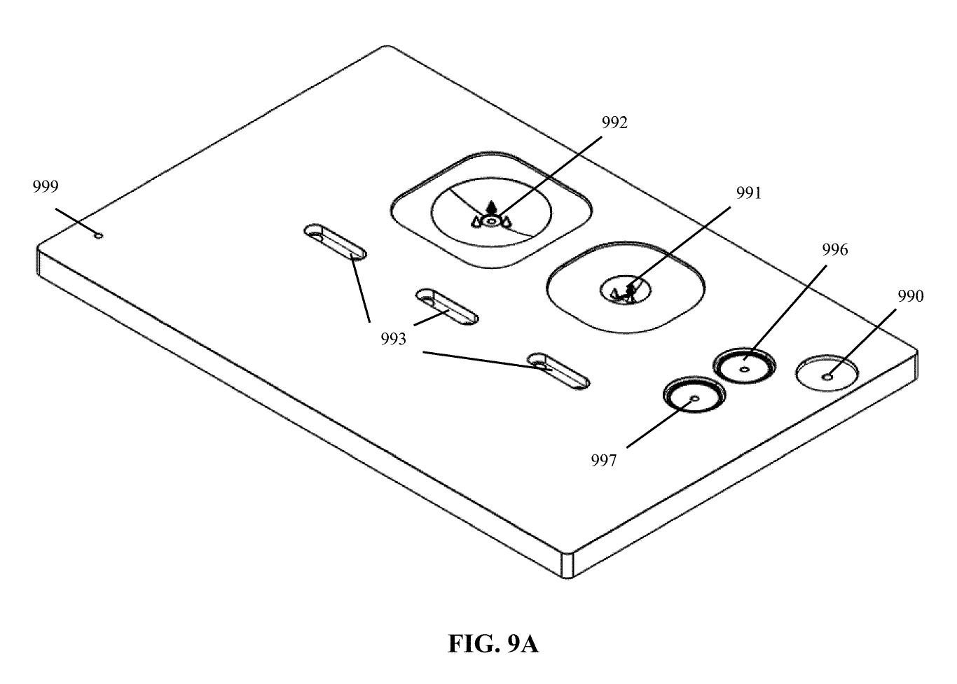

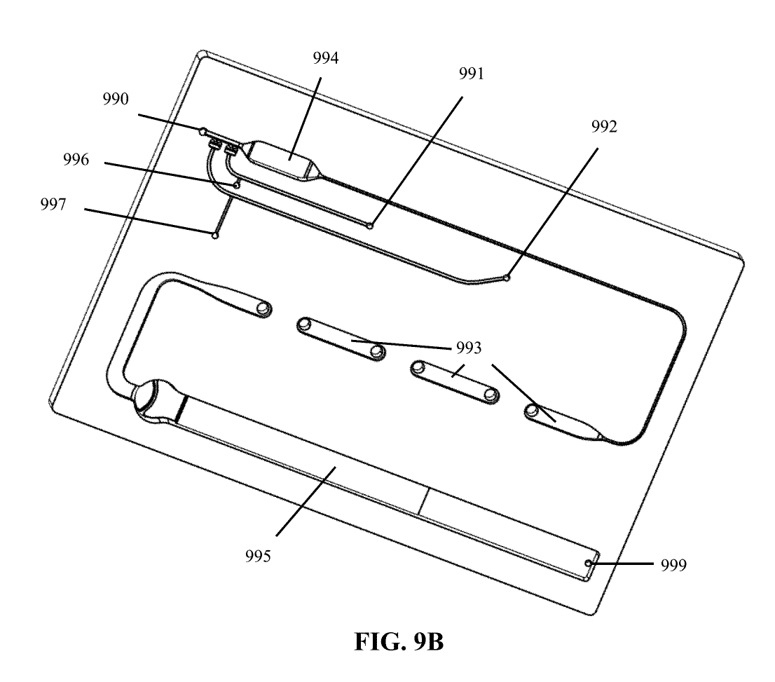

Certain embodiments are directed to a diagnostic chip designed for diagnosis of various disease, e.g., cutaneous leishmaniasis. FIG. 9A-9B illustrates a prototype device having DNA sample inlet 990, chamber with lyophilized master mix 991, blister containing rehydration buffer 996 connected to DNA amplification chamber 994 through a microfluidic channel, blister containing buffer to dilute amplification product 992, chambers 993 where mixing of dilution buffer and amplification product occur and flow towards lateral flow strip 995. Also illustrated are waste of overflow receptacle 997 and outlet 999.

### II. NUCLEIC ACID EXTRACTION DEVICE

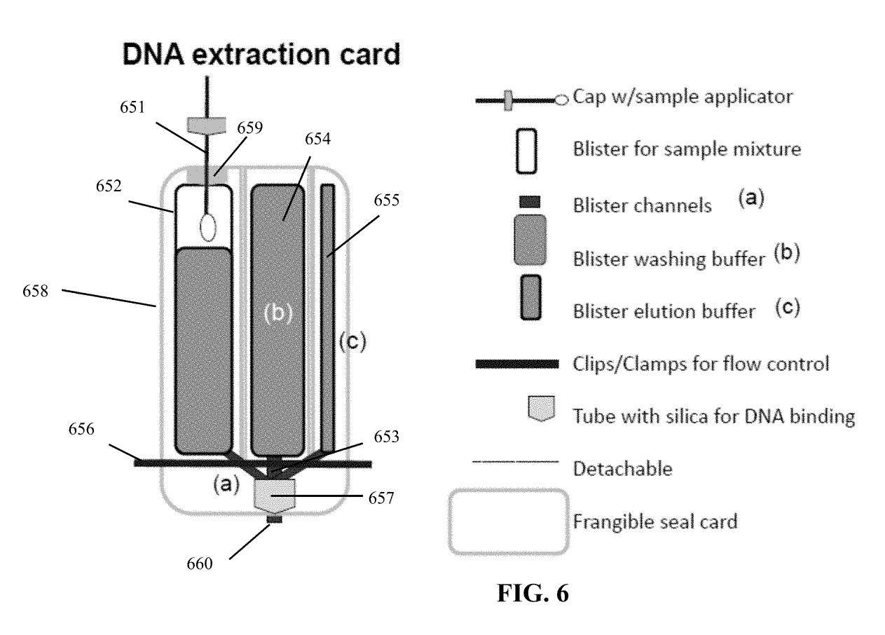

A sample analysis system can comprise the amplification device described above in conjunction with a nucleic acid extraction device. The nucleic acid extraction device can be configured to allow the transfer of a purified nucleic acid sample through the sample inlet and into the amplification reservoir of the amplification device described herein. As diagrammed in FIG. 6, one embodiment of a nucleic acid extraction device can comprise a body 658 having inlet 659 for inserting a sample to be processed and outlet 660 for elution of a purified nucleic acid composition or sample. The device can comprise at least three reservoirs (i) a sample reservoir 652, (ii) a washing buffer reservoir 654, and (iii) an elution buffer reservoir 655. In certain aspects the reservoirs are configured as blister packs containing an appropriate solution. Each of the reservoirs is independently in fluid communication with a nucleic acid binding component 657 of the device, which is in direct fluid communication with the outlet 660. A sample is introduced into the sample reservoir using a sample applicator 651. The sample is diluted and optionally lysed in the sample reservoir 652. Once the sample is processed in sample reservoir 652 the sample is expelled from sample reservoir 652 through nucleic acid binding component 657 of the device and out outlet 660 of the device. During transit through nucleic acid binding component 657 nucleic acids are bound and retained within nucleic acid binding component 657. Once sample reservoir 652 is emptied washing buffer reservoir 654 can be activated resulting in the expulsion of the washing buffer from the reservoir and through nucleic acid binding component 657 removing non-nucleic acid compounds and particles. Once the bound nucleic acid is washed, elution buffer reservoir 655 is activated and the elution buffer expelled from the reservoir through nucleic acid binding component 657. The elution buffer is such that the nucleic acid no long binds to the nucleic acid binding component and is eluted from the nucleic acid extraction card.

The eluted nucleic acid can be collected or inserted directly from the nucleic acid extraction card into the amplification device described herein. In certain aspects the nucleic acid binding component harbors silica for binding the nucleic acids present is a processed sample. Each of the reservoirs can be configured with a frangible seal that is broken once pressure is applied to the reservoir. A sample applicator can comprise a cap that is configured to seal the device once the applicator is position in the device. The applicator can be swab, a capillary, a micropipette, a scraper, or other apparatus useful for obtain a sample of cells, tissue, or the like for processing.

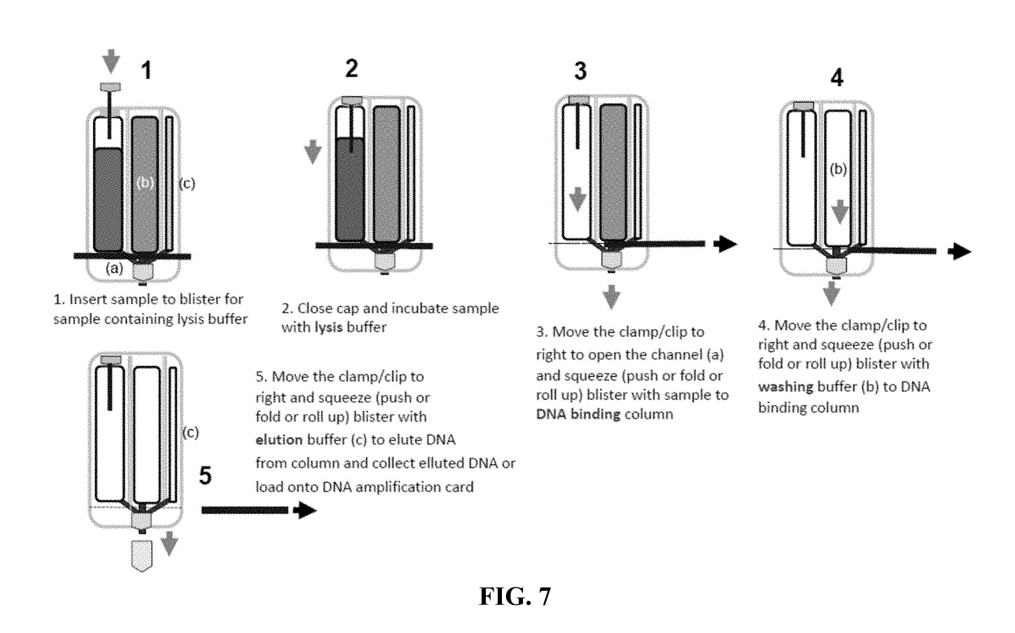

FIG. 7 provides an outline of one example of an extraction process using the nucleic acid extraction device described above. Step 1 is to insert the sample into the sample reservoir, in certain aspects the sample reservoir will contain a solution that lysis cells or organisms in the sample. The sample reservoir is closed and incubated for an appropriate time at an appropriate temperature. Once incubated the sample reservoir is opened or ruptured and the contents expelled through the nucleic acid binding component. In certain aspects a clip or clamp is present at the outlet of a reservoir. The clip can be moved to remove constriction of the reservoir to allow fluid to be evacuated from a reservoir. In other aspects a frangible seal is broken to allow fluid flow. Once the sample reservoir contents are passed through the nucleic acid binding component the washing buffer reservoir is opened and the content allowed to flow through the nucleic acid binding component providing a washing step for nucleic acid bound to the binding component. Once washed the elution reservoir is opened and the contents passed through the nucleic acid binding component to elute the bound nucleic acid from the nucleic acid extraction device. In certain aspects the nucleic acid is DNA, preferable genomic DNA.

### III. REAGENTS AND REACTIONS

The device can contain or can be capable of receiving reagents to amplify or detect a target nucleic acid, or to modify an amplification mixture for detection of a target nucleic acid. The reagents can include, but are not limited to, water, buffers, chemical reagents, enzymes (in either liquid or lyophilized form), nucleotides, primers, probes, pigments, antibodies, antigens, fluorophores, dyes, etc. The reagents can include reagents used for an isothermal amplification, more specifically recombinase polymerase amplification (RPA). The RPA reagents generally include, without limitation, the fluorophore probes, nucleotides, DNA polymerase, primers, recombinase, DNA binding proteins, ATP, phosphocreatine, creatine kinase, crowding agents, recombinase loading agents and the like.

The reagents can be incorporated during the manufacturing process and/or added to the device after manufacturing. The reagents can be located or placed alone or in combinations in the reservoirs of the device. Different reagents or combinations of reagents can be placed in different reservoirs in the device.

The device can be capable of amplifying nucleic acids in a sample. The nucleic acid amplification process can be initiated and completed by sequentially moving reagents or the sample from a reservoir to the next reservoir. The amplification can be done through an isothermal recombinase polymerase amplification. The reservoirs are capable of containing an optimal volume of fluid and/or reagents to perform the amplification and/or detection of a target nucleic acid.

### IV. NUCLEIC ACID DETECTION

The device can contain an indicator strip or detection region capable of detecting the presence of a target nucleic acid. The detection region can indicate the presence of target nucleic acid by a visual or other detectable signal. As a non-limiting example, the detection region can indicate the presence of the nucleic acid by changing colors, producing light, producing magnetic waves, producing radiation, changing shape, dissolving, providing an audible signal, changing conductivity, causing a chemical reaction, etc. In certain aspects a colored band or spot appears as an indication of the presence of the nucleic acid. The detection region can be a lateral flow strip. In certain aspects the detection region can be a lateral flow immunochromatographic test strip. The detection region can be included in a device described herein. In certain aspects the detection region can be included in a chamber or reservoir with a viewing window.

In certain aspects an amplification product can be introduced directly to the detection region from an amplification reservoir. In other aspects the amplification mixture can be diluted prior to or during transfer to the detection region.

The following examples of a nucleic acid amplification card and a DNA extraction card as well as the related figures are included to demonstrate preferred embodiments of the invention. It should be appreciated by those of skill in the art that the examples or figures represent devices and techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention.

Nucleic Acid Amplification and Identification Device. In one embodiment the device will consist of molded, flexible plastic that has multiple embedded reservoirs and “frangible seal” technology to temporarily block fluid flow from some of the reservoirs. The reservoirs can be designed to hold a specific volume or be empty or contained buffers, chemical reagents, and enzymes (which can be in either liquid or lyophilized form) that can be incorporated during the manufacturing process. A sample inlet or loading port can provide for loading a sample suspected of containing a target nucleic acid into the sample or amplification reservoir of the device. The nucleic acid amplification process can be initiated and completed by sequentially moving the amplification reagents to the amplification reservoir, and the amplified sample from the amplification reservoir to the processing reservoir to the detection region of the device by using manual pressure applied to the outside of the flexible molded plastic device. By applying pressure to the outside of the reservoir the seal of a reservoir under pressure will be broken to allow the flow of material through the channels from one reservoir to another. The fluid reaction mix will then be moved on to the next chamber in the same way by manual application of pressure to the reservoir. Flow from some or all reservoirs will be made unidirectional through, but not limited to, selective manual pressure (pushing the fluid in the correct direction), incorporation of one-way valves in the channels during the manufacturing process, or by external restriction achieved by a method such as simply folding the device and/or applying or removing an external clamp to restrict flow to a single channel can also be used.

In one method of using the device, each of the embedded reservoirs can have an optimal retention time and can hold an optimal volume. In the final step the reaction mix will be moved into a chamber or detection region containing an indicator that indicates the presence of a target nucleic acid. The detection region can comprise a lateral flow immunochromatographic test strip and have a transparent window in which the positivity or negativity of the detection region can be viewed or detected (e.g. presence or absence of a detectable band on a test strip). A general, non-limiting configuration of the device is shown in FIG. 1 to FIG. 5.

To test the ability of the nucleic acid amplification agents, immunochromatographic test strip, optimal volumes, and optimal reaction times the inventors first targeted identification of Leishmania spp. and Plasmodium spp. pathogens in clinical and control samples. Leishmania are a group of parasites that produce disfiguring cutaneous lesions or life-threatening disease (visceral leishmaniasis). Malaria, which is produced by Plasmodium spp. (P. falciparum and P. vivax) account for the highest mortality rates of all parasitic diseases in tropical countries.

The methods described herein using isothermal recombinase polymerase amplification and a lateral flow immunochromatographic test strip can be inexpensively used at the POC and are capable of identifying the presence of Leishmania spp. and Plasmodium spp. pathogens with surprising accuracy, sensitivity, and in a short amount of time. Specifically, it was found that the method described herein is highly specific, has similar sensitivity to PCR, is user friendly, is affordable, and can be used at the point of care. See U.S. patent application Ser. No. 14/993,407 filed Jan. 12, 2016, which is incorporated here by reference in its entirety. The inventors have subsequently targeted additional pathogens including Trypanosoma, Cryptosporidium, Giardia, and Zika virus using specific primers and probes that targeted highly repeated gene sequences of these different pathogens. This example can be seen as a proof of concept, as the diagnostic platform disclosed herein can be applicable to a larger number of infectious agents.

Certain embodiments of the device described herein is built upon two main components, a molecular component and a frangible seal technology component. The Molecular Component used specific primers and probes that were designed for this technology. In these examples, the DNA amplification was based on isothermal recombinase polymerase amplification (RPA), that has shown great potential for diagnosing parasitic, bacterial, or viral infections.

In a non-limiting example of the frangible seal technology component, the component can use a flexible plastic device that contains multiple embedded reservoirs designed to hold distinct reagent volumes. Buffer, chemical reagents, or enzymes (in either liquid or lyophilized form) can be incorporated during the manufacturing process and retained in the reservoirs. The reservoirs can be interconnected with channels between the reservoirs and the content of the reservoirs can be kept separate by frangible seals. A sample inlet or loading port can allowed the delivery of nucleic acid into the sample reservoir. In certain aspects the nucleic acid can be a purified nucleic acid. The movement of reagents can be accomplished by manual pressure applied to the outside of the flexible molded plastic device to break the frangible seal and move the reagents. The reaction mix can be moved into a chamber containing a lateral flow test strip, i.e., a detection region. The chamber or detection region can have a transparent window in which the positivity or negativity of the test strip can be determined with the naked eye (presence or absence of a detectable band on the test strip). See FIGS. 1, 2, and 3 below.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Efficacy of Recombinase Polymerase Amplification to Diagnose Trypanosoma cruzi Infection in Dogs with Cardiac Alterations from an Endemic Area of Mexico

A. Results

ELISA was positive in 96.6% (85/88) of dogs, confirming the frequent contact with infected triatomine bugs in the city of Mérida. All the dogs included in the study were positive by qPCR. The three dogs that were serologically negative resulted positive by qPCR and RPA-LF (dogs No. 54, 58 and 74; Table 1).

TABLE 1
Molecular and serological results and summary of cardiological findings in dogs form the city of Mérida, Mexico.

| | | | ELISA | | |
|---|---|---|---|---|---|
| Dog no. | qPCR Ct | RPA-LF | absorbance | Cardiac alteration | Compatible with Chagas disease |
| 1 | 22.62 | Positive | 0.546 | DVD, AVB | Acute-indeterminate phase |
| 2 | 24.44 | Positive | 0.707 | DVD | |
| 3 | 23.56 | Positive | 0.628 | DVD | |
| 4 | 22.42 | Positive | 0.54 | DVD | |
| 5 | 23.82 | Positive | 0.522 | DVD | |
| 6 | 22.70 | Positive | 0.902 | DVD | |
| 7 | 24.84 | Positive | 0.533 | DVD | |
| 8 | 23.02 | Positive | 0.519 | DVD | |
| 9 | 24.04 | Positive | 2.823 | AVB | Acute-indeterminate phase |
| 10 | 24.39 | Positive | 0.747 | CH | Indeterminate-chronic phase |
| 11 | 23.37 | Positive | 0.502 | DVD | |
| 12 | 23.67 | Positive | 0.531 | DVD, sinus blockade | |
| 13 | 24.55 | Positive | 0.538 | DVD | |
| 14 | 26.55 | Positive | 0.978 | N/A | |
| 15 | 25.86 | Positive | 0.497 | VP | |
| 16 | 25.65 | Positive | 0.293 | DVD, AVB | |
| 17 | 30.13 | Positive | 0.275 | AVB, CH | Acute-indeterminate phase |
| 18 | 27.38 | Positive | 0.418 | VP, CH | Indeterminate-chronic phase |
| 19 | 29.43 | Negative | 0.303 | DVD, CH | Indeterminate-chronic phase |
| 20 | 29.15 | Positive weak | 0.434 | VP | |
| 21 | 28.10 | Positive weak | 0.571 | VP | |
| 22 | 26.40 | Negative | 0.413 | DVD | |
| 23 | 28.28 | Negative | 0.885 | CH | Indeterminate-chronic phase |
| 24 | 27.81 | Positive | 0.250 | VP, CH | Indeterminate-chronic phase |
| 25 | 28.36 | Positive | 0.403 | DVD | |
| 26 | 29.24 | Negative | 0.699 | VP, sinus blockade | |
| 27 | 30.04 | Positive | 0.357 | VP, CH | Indeterminate-chronic phase |
| 28 | 28.79 | Negative | 0.220 | Sinus blockade | |
| 29 | 25.35 | Positive | 2.754 | VP, CH, AVB | Acute-indeterminate phase |
| 30 | 25.33 | Positive | 0.394 | VP, CH | Indeterminate-chronic phase |
| 31 | 27.31 | Positive | 2.756 | VP, CH | Indeterminate-chronic phase |
| 32 | 26.13 | Negative | 0.684 | VP | |
| 33 | 26.97 | Positive | 2.718 | AVB, CH | Acute-indeterminate phase |
| 34 | 27.71 | Positive | 0.379 | VP | |
| 35 | 26.69 | Positive | 0.733 | VP, CH | Indeterminate-chronic phase |
| 36 | 25.65 | Positive | 0.560 | VP, CH | Indeterminate-chronic phase |
| 37 | 23.61 | Positive | 0.525 | N/A | |
| 38 | 23.76 | Positive | 0.329 | VP, CH | Indeterminate-chronic phase |
| 39 | 23.02 | Positive | 0.230 | DVD | |
| 40 | 24.00 | Positive | 0.950 | N/A | |
| 41 | 24.25 | Positive | 0.243 | CH | |
| 42 | 23.88 | Positive | 0.931 | DVD | |
| 43 | 23.60 | Positive | 0.341 | CH | Indeterminate-chronic phase |
| 44 | 22.78 | Positive | 0.607 | VP | |
| 45 | 23.97 | Positive | 0.254 | VP, AVB | Acute-indeterminate phase |
| 46 | 23.36 | Positive | 0.55 | CH, ARR | Indeterminate-chronic phase |
| 47 | 22.79 | Positive | 0.285 | CH, ARR | |
| 48 | 21.84 | Positive | 0.509 | CH | Indeterminate-chronic phase |
| 49 | 23.41 | Positive | 0.38 | N/A | |
| 50 | 22.37 | Positive | 0.473 | VP, CH | Indeterminate-chronic phase |
| 51 | 23.23 | Positive | 0.347 | AVB | Acute-indeterminate phase |
| 52 | 22.92 | Positive | 2.625 | VP, CH | Indeterminate-chronic phase |
| 53 | 24.98 | Positive | 0.512 | CH | Indeterminate-chronic phase |
| 54 | 26.10 | Positive | 0.176 | VP | |
| 55 | 24.67 | Positive | 0.298 | DVD | |
| 56 | 25.90 | Positive | 0.234 | VP, CH | Indeterminate-chronic phase |
| 57 | 23.13 | Positive | 2.813 | CH | Indeterminate-chronic phase |
| 58 | 25.27 | Positive | 0.205 | AVB, CH | Indeterminate-chronic phase |
| 59 | 23.81 | Positive | 0.204 | VP | |
| 60 | 23.80 | Positive | 0.318 | VP | |
| 61 | 24.49 | Positive | 0.531 | N/A | |
| 62 | 24.97 | Positive | 0.236 | VP, CH | Indeterminate-chronic phase |
| 63 | 23.30 | Positive | 0.265 | N/A | |
| 64 | 23.45 | Positive | 0.52 | VP | |
| 65 | 29.39 | Positive | 0.221 | VP, CH | Indeterminate-chronic phase |
| 66 | 28.93 | Positive | 0.546 | VP | |
| 67 | 23.46 | Positive | 0.31 | ARR (sinusal) | |
| 68 | 24.48 | Positive | 0.225 | ARR (sinusal) | |
| 69 | 22.94 | Positive | 2.806 | ARR (ventricular) | Acute phase |
| 70 | 28.55 | Positive | 0.327 | VP, CH | Indeterminate-chronic phase |
| 71 | 25.23 | Positive | 0.251 | VP, CH | Indeterminate-chronic phase |
| 72 | 21.71 | Positive | 0.359 | VP, CH | Indeterminate-chronic phase |
| 73 | 23.88 | Positive | 0.196 | CH | Indeterminate-chronic phase |
| 74 | 26.25 | Positive | 0.144 | VP, CH | Indeterminate-chronic phase |
| 75 | 26.22 | Positive weak | 0.457 | VP, CH | Indeterminate-chronic phase |
| 76 | 24.65 | Positive | 0.412 | CH, VP | Acute phase |
| 77 | 23.61 | Positive | 2.206 | VP | |
| 78 | 25.39 | Positive | 0.327 | VP, CH | Indeterminate-chronic phase |
| 79 | 22.30 | Positive | 0.251 | ARR | |
| 80 | 22.06 | Positive | 0.233 | VP | |
| 81 | 21.62 | Positive | 0.316 | VP, ARR | |
| 82 | 21.98 | Positive | 0.264 | VP, CH, AVB | Acute-indeterminate phase |
| 83 | 23.00 | Positive | 0.23 | VP | |
| 84 | 23.17 | Positive | 0.684 | CH, VP | Acute-indeterminate phase |
| 85 | 23.58 | Positive | 0.403 | ND | |
| 86 | 23.78 | Positive | 0.289 | ND | |
| 87 | 23.93 | Positive | 0.294 | ND | |
| 88 | 24.16 | Positive | | ND | |
| Negative control | ≥38.4 | | Cut-off 0.217 | | |

Only principal cardiological findings were annotated: other cardiac alterations are not included.
ARH, arrhythmia; AVB, atrium-ventricular blockade; CH, cardiac hypertrophy; Ct, cycle threshold; DVD, degenerative valvular disease; ELISA, enzyme-linked immunosorbent assay; N/A, other unrelated pathology; ND, not done; RPA-LF, recombinase polymerase amplification-lateral flow; VP, valvular pathology.

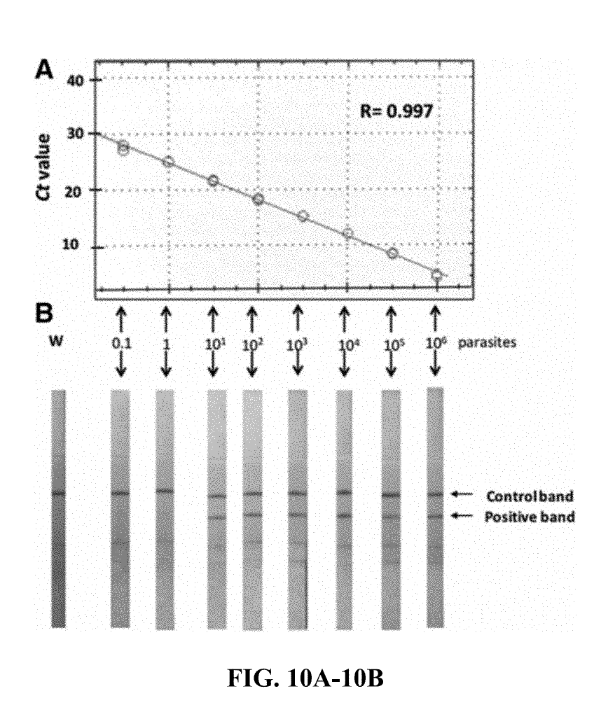

The initial analytical sensitivity indicated that RPA-LF amplified T. cruzi DNA in samples containing 1-2 parasites per reaction, which corresponded to Ct values of 27 or 26 in the real-time PCR used as gold standard (FIG. 10). Serial two fold dilutions of T. cruzi epimastigotes showed that RPA-LF had 95% (19/20) repeatability at concentrations of two parasites per reaction.

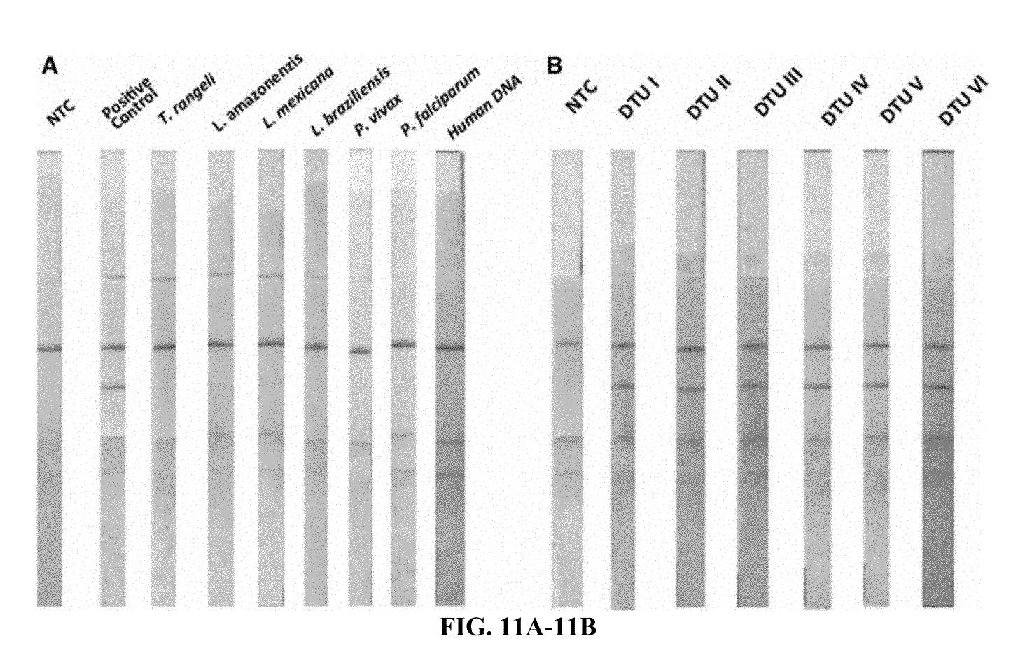

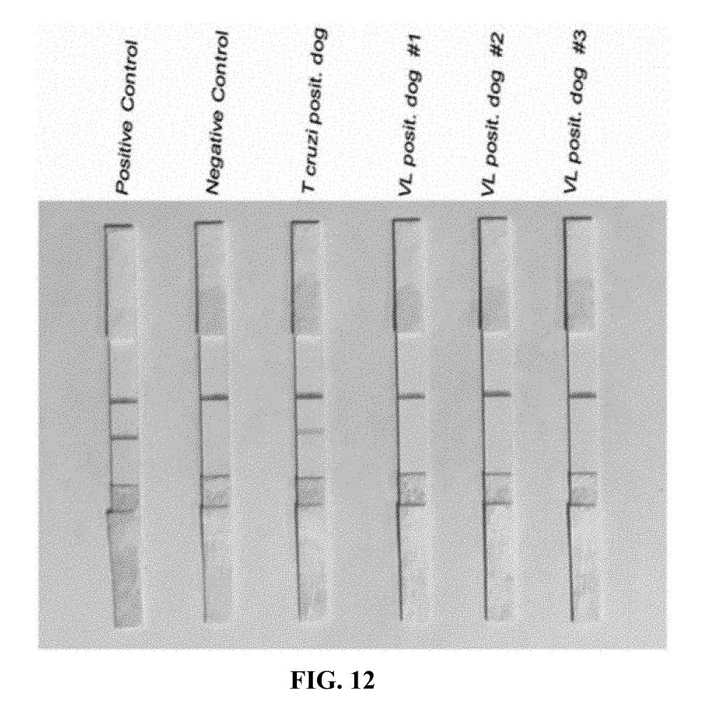

RPA-LF showed good specificity when run in the presence of human DNA or other protozoan parasites (Leishmania spp. and Plasmodium spp.) (FIG. 11A and FIG. 12). There was no cross-reactivity with the closely related, but nonpathogenic T. rangeli, which infects different mammals, including dogs and humans. Of epidemiological relevance was the capacity of RPA-LF to amplify all DTU's (I-VI) of T. cruzi that circulate in domestic and extradomestic environments of different countries (FIG. 11A).

The diagnostic efficacy of RPA-LF was determined using DNA from retrospective blood samples obtained from 88 dogs inhabiting the city of Mérida that came for consultation to the Faculty of Veterinary Medicine and Animal Science (UADY), Mérida. Most of these infected dogs (confirmed by qPCR; Table 1) presented cardiomyopathies, but not all of them were compatible with Chagas disease. The RPA-LF detected T. cruzi DNA in 82 of the 88 samples, reaching a sensitivity of 93.2% (95% confidence interval 87.2-98.1) and excellent agreement with qPCR (Cohen's Kappa test=0.963). Four of the six dogs that resulted negative by RPA-LF had low parasite burden as indicated by the high Ct values (≥28.28) of qPCR (Table 1). No RPA-LF false positive results were found when DNA samples from uninfected dogs or blood spiked with unrelated pathogens were included in the RPA-LF runs.

B. Materials and Methods

Study Sites.

All dogs were from Mérida, Yucatan, Mexico (19′30″ and 21′35″N latitude, and 87′30″ and 90′24″ W longitude). Housing in many neighborhoods favors triatomine infestation and sustains active T. cruzi transmission (Guzman-Tapia et al. 2007, Jimenez-Coello et al. 2010).

Dog Samples.

88 naturally infected dogs brought for consultation at the Faculty of Veterinary Medicine and Animal Science of the Autonomous University of Yucatan (UADY), Mérida were evaluated. The reason for consultation was the existence of cardiomyopathies.

Blood samples were obtained by venipuncture of the cephalic vein. Three milliliters of whole blood was collected in PAX-gene (cat. no. 761125; BD-Qiagen) to preserve DNA until purification. An additional 3 mL sample without anticoagulant was obtained using sterile Vacutainer tubes and centrifuged at 2000 g for 10 min to collect serum. Both, purified DNA and sera were stored at −20° C. until use (Jimenez-Coello et al. 2015).

Serology for Anti-T. cruzi IgG Detection.

Serum samples were evaluated using an enzyme-linked immunosorbent assay (ELISA; Chagatest-ELISA recombinant v.4.0 kit; Wiener Laboratories S.A.I.C.). This ELISA test detects antibodies to six recombinant proteins expressed in T. cruzi. The assay was carried out following the manufacturer's recommendations, except for the second antibody that was replaced with goat anti-dog IgG conjugated with horseradish peroxidase (HRP; sc-2433; Santa Cruz Biotechnology). Briefly, 96-well plates were coated with recombinant proteins, and then sequentially incubated with 20 μL of serum samples (1:80 dilution) in phosphate buffer (137 mM NaCl, 2.7 mM KCl, 4.3 mM Na₂HPO₄, and 1.4 mM KH₂PO₄, pH 7.4) and HRP-conjugated dog anti-IgG (1:5000 dilution). Color was developed with tetramethylbenzidine and hydrogen peroxide substrates, and the reaction was stopped by acidification of the reaction medium. The optical density (OD) was read at 450 nm in an xMark™ microplate absorbance spectrophotometer (Bio-Rad, Hercules, Calif.). The cutoff value of 0.217 was determined from the mean value of the negative control sera ±3 standard deviations (Medina 2002).

DNA Extraction.

DNA was purified from whole blood samples according to Jalal et al. (2004). Subsequently, the protocol of the commercial kit was used, DNeasy Blood and tissue kit (69504; Qiagen, Germantown, Md.), following the manufacturer instructions. Total DNA was examined for quality (OD260/OD280 ratio of 1.7-2.0) and quantity ([OD260−OD320]x·50 μg/mL) using a DU® 800 ultraviolet/visible spectrophotometer.

Quantitative PCR.

The quantitative PCR (qPCR) was performed with Sso-Advanced Universal SYBR Green Supermix (172-5271; Bio-Rad) and oligonucleotides (TCZ-F 5′-GATCTTGCCCACAMGGGTGC-3′ (SEQ ID NO:1) and TCZ-R 5′-CAAAGCAGCGGATAGTTCAGG-3′) (SEQ ID NO:2) as previously described (Schijman et al. 2011). Samples were evaluated by qPCR for glyceraldehyde-3-phosphate dehydrogenase (GAPDH) gene to detect the potential presence of inhibitors in the reaction mix and to use equal amounts of DNA in all reactions.

Duplicate samples of total DNA (4 μL) were used as template for qPCR to estimate the T. cruzi loads. The standard curve was prepared using T. cruzi epimastigotes, clone CL Brener, TcVI as described by Bua et al. (2012). Briefly, 1 mL of blood from a seronegative dog was spiked with 10⁷ parasites/mL and the total DNA was extracted. Tenfold serial dilutions of the extracted DNA, corresponding to 10⁶-0.1 parasite/mL (corresponding to 1×10³ to 1×10⁴ parasite equivalents per assay), were used for running the qPCR (Cencig et al. 2011). Total DNA from healthy dogs was used as negative controls. All experiments included the parasite standard curve, DNA from negative controls, and no-template DNA control. The parasite loads were determined by linear regression analysis of the cycle threshold (Ct) values of the test samples against Ct values of the standard curve based on known amounts of T. cruzi DNA. The results are presented as Ct values and T. cruzi per mL. A melting curve analysis was performed in all qPCR-positive samples to confirm the presence of only one peak in each sample.

RPA and Lateral Flow Reading.

Design of primers and probe. The primer sets were designed for T. cruzi and are 30-32 nucleotides long and target conserved sequences identified by computational alignment of T. cruzi satellite sequences reported in GenBank. Primers were designed with 40-60% GC content, few direct/inverted repeats, and absence of long homopolymer tracts. The inventors focused on conserved regions and to a lesser extent on regions with moderate variability, obtaining a 146 base pair (bp) RPA amplicon in agarose gels. To enable detection by lateral flow (LF; as described below), the reverse primer was biotinylated at the 5′ end. A 45 bp conserved internal probe was designed (Biosearch Technologies, Petaluma, Calif.) that included FAM (5′-carboxy fluorescein amidite) at the 5′ end, an internal dSpacer, and a SpacerC3 in the 3′ end, as suggested by the manufacturer (TwistDx). Therefore, the primer set and probe used in this work were as follows: Fw03 5′ GCTGCACTCGGCGGATCGTTTTCGAG 3′ (SEQ ID NO:3); Rev 05 5′ GTTTGGTGTCCAGTGTGTGAACACGCAAACA 3′ (SEQ ID NO:4); and Probe-FAM-5′-GCACCACACGTTGTGGTCTAAATTTTTGTTTCGAATTATGAATGG-3′ (SEQ ID NO:5).

RPA Reaction and LF Reading.

The amplification mixture per reaction comprised the following: (1) forward primer (0.89 mM), (2) biotinylated reverse primer (0.89 mM), (3) FAM-labeled probe (0.22 mM), (4) magnesium acetate (1.25 μL), and (5) the rehydrated cocktail (14.5 μL; Twist amp nfo RPA kit; TwistDx). Template DNA (5-25 ng/μL) of parasite-spiked samples or clinical samples was immediately added to the mixture and subjected to amplification at 40° C. for 30 min using a dry bath. The RPA product was diluted at 2% in the dipstick assay buffer and 100 μL was placed in a 1.5 mL microtube. The bottom tip of the LF strip (Ustar Biotechnologies, Hangzhou, China) was then immersed in the sample, making the amplification product run upwards by capillarity. Parasite amplification was confirmed with the naked eye after 5 min by the appearance of the test band in the lower part of the strip. The positive test band is produced when anti-biotin antibodies immobilize the amplified DNA, which contains the biotinylated reverse primers. The gold particles in the strip, which are covered with mouse anti-FAM antibodies, bind to the FAM-labeled probe making the test band visible. The reaction was validated by the appearance of the control band in the upper part of the strip. This band appears upon the immobilization of excess free gold particles (which are covered with mouse antibodies) by means of anti-mouse antibodies. Positive and negative controls were included in each round of RPA-LF.

Sensitivity and Specificity of RPA-LF.

The sensitivity of the test was established using normal dog blood spiked with serial dilutions (10⁶-0.1 parasite/mL) of T. cruzi epimastigotes of clone CL Brenner, TcVI, as template. To determine the specificity, the RPA-LF was run using Trypanosoma rangeli, Leishmania mexicana, Leishmania braziliensis, Leishmania infantum, Plasmodium falciparum, and Plasmodium vivax together with the six T. cruzi discrete typing units (DTUs I-VI). In silico analysis showed that the RPA primers did not align with Trypanosoma caninum. This recently described that nonpathogenic trypanosome was isolated from dog skin only in Brazil (Madeira et al. 2014).

### REFERENCES

Aparicio-Burgos et al. PLoS Negl Trop Dis 2015; 9:e0003625.
Bahia et al. Rev Soc Bras Med Trop 2002; 35:339-345.
Barr, Canine Chagas' disease (American trypanosomiasis) in North America. Vet Clin North Am Small Anim Pract 2009; 39:1055-1064, v-vi.
Barr et al. J Am Vet Med Assoc 1989; 195:1237-1241.
Beard et al. Emerg Infect Dis 2003; 9:103-105.
Beaumier et al. Vaccine 2016; 34:2996-3000.
Bisio et al. Trans R Soc Trop Med Hyg 2011; 105:543-549.
Bua et al. Trans R Soc Trop Med Hyg 2012; 106:623-628.
Caldas et al. Trop Med Int Health 2013; 18:75-84.
Cardinal et al. Am J Trop Med Hyg 2006; 75:753-761.
Castillo-Neyra et al. Prev Vet Med 2015; 120:349-356.
Cencig et al. PLoS Negl Trop Dis 2011; 5:e1216.
Curtis-Robles et al. Am J Trop Med Hyg 2017; 96:805-814.
Degrave et al. Mol Biochem Parasitol 1988; 27:63-70.
Dias et al. Epidemiol Sery Sau'de 2016; 25:7-86.
Enriquez et al. Acta Trop 2013; 126:211-217.
Enriquez et al. Vet Parasitol 2016; 223:186-194.
Gan et al. Lab Chip 2014; 14:3719-3728.
Govindarajan et al. Lab Chip 2012; 12:174-181.
Gurtler and Cardinal, Acta Trop 2015; 151:32-50.
Gurtler et al. Am J Trop Med Hyg 1996; 55:24-31.
Gurtler et al. Parasitology 2007; 134:69-82.
Guzman-Tapia et al. Vector Borne Zoonotic Dis 2007; 7:597-606.
Jalal et al. Clin Microbiol Infect 2004; 10:937-939.
Jimenez-Coello et al. J Venom Anim Toxins Incl Trop Dis 2015; 21:37.
Jimenez-Coello et al. Transbound Emerg Dis 2010; 57:33-36.
Lauricella et al. Mem Inst Oswaldo Cruz 1998; 93:501-507.
Machado et al. Am J Trop Med Hyg 2001; 65:958-965.
Madeira et al. J Parasitol 2014; 100:231-234.
Mahalanabis et al. Biomed Microdevices 2010; 12:353-359.
Medina, Revista de la Facultad de Ciencias de la Salud 2002; 6:4.
Petersen et al. Parasitol Res 2001; 87:208-214.
Quijano-Hernandez et al. Vaccine 2013; 31:2246-2252.
Schijman et al. PLoS Negl Trop Dis 2011; 5:e931.
Stanaway and Roth, Glob Heart 2015; 10:139-144.
Sturm et al. Mol Biochem Parasitol 1989; 33:205-214.
Vargas et al. Mol Biochem Parasitol 2004; 138:131-141.
Vitt et al. J Vet Intern Med 2016; 30:1210-1215.
WHO. Weekly epidemiological record (WER). Geneva: World Health Organization, 2015:33-44.
Castellanos-Gonzalez et al. Am J Trap Med Hyg. 2015 Nov. 4; 93(5):970-5.
Crannell et al. Am j Trap Med Hyg. 2015 March; 92(3):583-7.
Crannell et al. Anal Chern. 2014 Mar. 4; 86(5):2565-71.
Nair et al Am J Trap Med Hyg. 2015 September; 93(3):591-5.
Saldarriaga et al. PLoS Negl Trop Dis. 2016 Apr. 26; 10(4):e0004638

1. A self-contained nucleic acid amplification device for detecting a target nucleic acid in a sample, the device comprising:
a plurality of reservoirs and fluid channels connecting the reservoirs, wherein:
(i) a rehydration reservoir fluidly connected to a reagent reservoir that contains nucleic acid amplification reagents;
(ii) an amplification reservoir fluidly connected to the reagent reservoir, the amplification reservoir being configured to receive (a) a sample comprising nucleic acid and (b) reconstituted nucleic acid amplification reagents from the reagent reservoir; and
(iii) a detection region fluidly connected to the amplification reservoir, the detection region being configured for visual detection of amplified nucleic acids.

2. The device of claim 1, further comprising: (iv) a processing reservoir fluidly connected to the amplification reservoir, the processing reservoir containing nucleic acid detection reagents, and, optionally, (v) a product dilution blister fluidly connected to the amplification reservoir and receives a volume of amplification product from the amplification reservoir;

3. The device of claim 1, wherein one or more of the rehydration reservoir, reagent reservoir, and amplification reservoir are configured as a laterally extended blister.

4. The device of claim 1, wherein the reagent reservoir contains at least a nucleic acid polymerase enzyme, nucleotides, and nucleic acid primers.

5. The device of claim 1, wherein the nucleic acid amplification reagents are isothermal amplification reagents.

6. The device of claim 5, wherein the isothermal amplification reagents are recombinase polymerase amplification (RPA) reagents.

7. The device of claim 1, wherein at least one reservoir is sealed by a frangible seal.

8. The device of claim 7, wherein the frangible seal is configured to be broken by the application of pressure to a reservoir.

9. The device of claim 1, wherein at least fluid channel is reversibly closed by a valve, an external clamp, or a fold in the device.

10. The device of claim 1, wherein the device is configured for directional fluid flow from the rehydration reservoir to the detection region.

11. The device of claim 1, further comprising a removable clamp positioned over and closing a fluid channel.

12. The device of claim 1, further comprising a fold-line positioned across a fluid channel and configured to close the fluid channel when the device is folded along the fold-line.

13. The device of claim 1, wherein the amplification reservoir further comprises a sample inlet.

14. The device of claim 13, wherein the sample inlet comprises a luer-lock syringe adaptor, a screw-on cap, or a hinged snap cap.

15. The device of claim 1, wherein the detection region comprises a lateral flow immunochromatographic strip.

16. The device of claim 1, wherein the detection region comprises a viewing window.

17. A nucleic acid amplification system for detecting a target nucleic acid in a sample, the system comprising:
(a) a nucleic acid extraction card comprising:
(i) a body having an inlet for insertion of a sample;
(ii) a sample reservoir accessible through the inlet and fluidly coupled to a nucleic acid binding component which is fluidly coupled to an outlet,
(iii) a washing buffer reservoir fluidly coupled to the nucleic acid binding component,
(iv) an elution buffer reservoir fluidly coupled to the nucleic acid binding component; and
(b) a nucleic acid amplification card comprising
(i) a rehydration reservoir fluidly connected to a reagent reservoir that contains nucleic acid amplification reagents;
(ii) an amplification reservoir fluidly connected to the reagent reservoir, the amplification reservoir being configured to receive (1) a sample comprising nucleic acid and (2) reconstituted nucleic acid amplification reagents from the reagent reservoir;
(iii) a processing reservoir fluidly connected to the amplification reservoir, the processing reservoir containing nucleic acid detection reagents;
(iv) a product dilution blister fluidly connected to the amplification reservoir and receives a volume of amplification product from the amplification reservoir; and
(v) a detection region fluidly connected to the product dilution blister, the detection region being configured for visual detection of amplified nucleic acids;
wherein the nucleic acid extraction card outlet is configured to be removably connected to the nucleic acid amplification card inlet.

18. A method of using the self-contained nucleic acid amplification device of claim 1 to amplify and identify the presence of a nucleic acid with a specific sequence in a sample, the method comprising:
(a) inserting a sample comprising a nucleic acid into an amplification reservoir through a sample inlet;
(b) rehydrating amplification reagents contained in the reagent reservoir by expelling a rehydration solution from the rehydration reservoir into the reagent reservoir;
(c) combining the sample and the amplification reagents by expelling the rehydrated amplification reagents form the reagent reservoir into the amplification reservoir forming an amplification solution;
(d) incubating the amplification solution under conditions that result in amplification of a target nucleic acid producing an amplification product; and
(e) transferring the amplification product to a processing reservoir containing a detection reagent forming a detectable target nucleic acid; and
(f) transferring the detectable nucleic acid to a detection region where the detectable nucleic acid is visualized.

19. The method of claim 18, wherein the amplification reagents include nucleic acid polymerase, nucleic acid primer, and nucleotides.
